(19) United States
(12) Patent Application Publication
(10) Pub. No.: US 2023/0390260 A1
(43) Pub. Date: Dec. 7, 2023

(54) METHOD OF TREATMENT OF PAIN THROUGH BUCCAL ADMINISTRATION OF FLUNIXIN

(71) Applicant: Intervet Inc., Madison, NJ (US)

(72) Inventor: Julien Thiry, Nice (FR)

(73) Assignee: Intervet Inc., Madison, NJ (US)

(21) Appl. No.: 18/253,140

(22) PCT Filed: Nov. 24, 2021
(86) PCT No.: PCT/EP2021/082742

§ 371 Date: May 16, 2023

Related U.S. Application Data
(60) Provisional application No. 63/118,036, filed on Nov. 25, 2020.

(51) Int. Cl. A61K 31/44; A61K 9/00; A61K 47/22; A61K 47/10; A61K 47/34; A61K 47/14; A61P 29/00

(52) U.S. Cl. CPC A61K 31/44; A61K 9/006; A61K 47/22; A61K 47/10; A61K 47/34; A61K 47/14; A61P 29/00

(57) ABSTRACT

A buccal administration method of flunixin has been developed now to provide an easy to use and effective method of reducing pain in livestock animals (e.g. swine, cattle, sheep, goats, etc.).

### BACKGROUND OF THE INVENTION

In livestock operations, there is a concern for the well-being of animals in general and in particularly during certain activities that may produce pain to the animal. There is a need for methods to reduce pain during activities, such as castration, tail docking, removal of needle teeth and the like.

Flunixin is a nonsteroidal anti-inflammatory drug (NSAID). Typically, flunixin is available as the meglumine salt. Flunixin is used as an analgesic and for the treatment of pyrexia and alleviation of clinical signs of inflammation associated with mastitis and respiratory disease (BRD), as well as for the reduction of pain and swelling from acute inflammatory conditions, in bovine species. In swine, flunixin is indicated for swine respiratory disease. No flunixin containing veterinary medicinal products are marketed for pain management in swine in the USA.

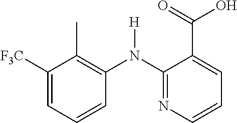 [SMILES: Cc1c(Nc2ncccc2C(=O)O)cccc1C(F)(F)F]

Currently, flunixin is available as an injectable solution, a transdermal solution or a paste. The use of these products varies depending indication and species.

The commercially available flunixin transdermal product is used to reduce pyrexia associated with bovine respiratory disease and acute mastitis in cattle. (see Health Products Regulatory Authority (HPRA) Publicly Available Assessment Report for a Veterinary Medicinal Product Finadyne Transdermal 50 mg/mL pour on solution for cattle, CRN 7023803, Jan. 23, 2017). The transdermal product contains 50 mg flunixin (equivalent to 83 mg flunixin meglumine), 150 mg pyrrolidone, 50 mg L-menthol, 500 mg propylene glycol dicaprylate/dicaprate NF, 0.20 mg FD&C Red No. 40, and glycerol monocaprylate NF qs (see Banamine label). This formulation is described in U.S. Pat. No. 9,006,727 B2.

Flunixin is also known in tablet which dissolves intraorally. See U.S. Ser. No. 10/022,361, WO2007061529 and WO2003066029 for example. These oral tablet products are difficult for use with animals as they are often not retained until complete oral dissolution is achieved.

None of these references discloses the buccal administration of flunixin.

### SUMMARY OF THE INVENTION

Young piglets can experience significant pain during common farming procedures, including castration, docking, or having needle teeth clipped, among others. Other animals also experience pain during similar procedures.

A buccal administration method of flunixin has been developed now to provide an easy to use and effective method of reducing pain in livestock animals (e.g. swine, cattle, sheep, goats, etc.).

A method of treating pain comprising buccal administration to an animal in need thereof a composition comprising flunixin or a salt thereof.

### DETAILED DESCRIPTION OF THE INVENTION

### Definitions

Buccal administration means to apply the drug to the buccal area of the cheek to diffuse through the oral mucosa and enter directly into bloodstream. It is considered a topical route of administration. Buccal administration allows for better bioavailability and a more rapid onset of action of some drugs compared to oral administration since the drug avoids metabolism by not passing through the digestive system.

Sublingual administration involves placing a drug under your tongue to dissolve and absorb into your blood through the tissue there.

Oral mucosa are tissues which line the mouth. The buccal mucosa are the tissues lining the cheeks.

Pharmaceutically acceptable carriers are substances that improve the selectivity, effectiveness and safety of drug administration. Pharmaceutically carriers are frequently employed to control the systemic release of a drug into a patient.

Transdermal composition is a pharmaceutical composition which is used to administer a drug across the skin.

Perineum is the area between the anus and the scrotum or vulva.

Banamine® Transdermal is a flunixin containing transdermal formulation whose composition is described in the table below:

TABLE 1

| Component | Purpose | % w/v |
|---|---|---|
| Flunixin Meglumine USP | API | 8.3 |
| (Flunixin free acid equivalent) | | (5.0) |
| Pyrrolidone EP | Solvent | 15.0 |
| (Soluphor P ex. BASF) | | |
| Levomenthol EP | Penetration | 5.0 |
| (l-Menthol USP) | Enhancer | |
| Propylene Glycol | Penetration | 50.0 |
| Dicaprylocaprate EP | Enhancer | |
| (Propylene Glycol | & | |
| Dicaprylate/Dicaprate NF) | Diluent | |
| (Miglyol 840 ex. Sasol) | | |
| Allura Red AC E129 | Color | 0.02 |
| (FD&C Red No. 40) | | |
| Glyceryl Monocaprylate EP | Bridging | Q.S. |
| (Mono- and Diglycerides NF) | Vehicle | (Approx. |
| (Imwitor 988 ex. Sasol) | | 20.7% LC) |

The composition and formation of flunixin transdermal formulations is described in U.S. Pat. No. 9,006,727 B2.

Banamine®-S Injectable Solution is an injectable flunixin contain product. Each milliliter of Banamine®-S Injectable Solution contains flunixin meglumine equivalent to 50 mg flunixin, 0.1 mg edetate disodium, 2.5 mg sodium formaldehyde sulfoxylate, 4.0 mg diethanolamine, 207.2 mg propylene glycol; 5.0 mg phenol as preservative, hydrochloric acid, water for injection q.s. (see Banamine®-S Injectable Solution product bulletin).

Banamine®-paste is non-steroidal anti-inflammatory drug (NSAID) approved for horses in the United States. The paste contains propylene glycol, carmellose sodium, maize starch and purified water. The paste is orally administered by inserting the nozzle of the syringe through the interdental space and depositing the required amount of paste on the back of the tongue by depressing the plunger.

Penetration enhancers are also referred to as absorption promoters or accelerants have several advantages increase in transdermal flux of in transdermal drug delivery in comparison with passive diffusion. They are normally painlessness and noninvasiveness. Examples of penetration enhancers are menthol, camphor, d-limonene, 1-8 Cineole, xylene, isopropyl myristate, propylene glycol dicaprylate/dicaprate, decanoic acid, decyl alcohol, oleic acid or mixtures thereof. Preferred are mixtures of menthol and propylene glycol dicaprylate/dicaprate.

Aprotic solvent is a solvent that has no O—H or N—H bonds. Examples of aprotic solvents are pyrrolidone solvents such as 2-pyrollidone or N-methyl-2-pyrrolidone or mixtures thereof, N,N-dimethylacetamide, N,N-dimethylforamide, DMSO, acetone, glycerol formal, ethyl lactate and glycol ethers such as ethylene glycol mono-ethyl ether, diethylene glycol mono-ethyl ether or dipropylene glycol mono-ethyl ether or mixtures thereof. Particularly, the aprotic solvent is a pyrrolidone solvent, preferable 2-pyrollidone.

Bridging vehicles are excipients that promote solubility between other formulation components. Examples are glyceryl monocaprylate EP (Mono- and Diglycerides NF).

A piglet is a small usually young swine.

An embodiment of the invention is a method of treating pain in an animal comprising buccal administration to an animal in need thereof of a composition comprising flunixin or a salt thereof.

In another embodiment, the composition is a transdermal composition.

In another embodiment, the composition further comprises one or more penetration enhancers and/or an aprotic primary solvent.

In another embodiment, the flunixin is flunixin meglumine.

In another embodiment, the penetration enhancers are menthol, propylene glycol dicaprylate/dicapratecaprate or mixtures thereof.

In another embodiment, the aprotic primary solvent is a pyrrolidone solvent, preferable 2-pyrollidone.

In another embodiment, the animal is a livestock animal

In another embodiment, the livestock animal is a pig, a goat, a sheep or a bovid.

In another embodiment, the animal is a piglet.

In another embodiment, the pain is a result of castration, tail docking or removal of needle teeth.

In another embodiment, the buccal administration is to the inside of the cheek of the animal.

An embodiment of the invention is a method of treating pain comprising buccal administration to an animal in need thereof a composition comprising
a) flunixin meglumine,
b) 2-pyrrolindone,
c) menthol,
d) propylene glycol dicaprylate/dicapratecaprate, and
e) glyceryl monocaprylate.

In another embodiment, the animal is a piglet.

### EXAMPLE

The administration of flunixin to manage pain in piglets has been investigated. A pharmacokinetic study demonstrated that adequate levels of flunixin can be achieved through buccal administration.

A pharmacokinetic study was conducted to compare the bioavailability of flunixin when administered buccally, intramuscularly, IV, and the transdermal routes via the perineum, ear canal, rectum, and midline in young piglets.

Piglets were administered either Banamine® Transdermal (buccal, perineum, ear canal, rectum, and midline) or Banamine®-S Injectable Solution (intravenous and intramuscular) at 5 to 6 days of age. Blood samples were collected from the piglets at 5 min, 15 min, 1 h, 2 h, 4 h, 8 h, 12 h, 24 h, 36 h, and 48 h for those treated with Banamine® Transdermal, and at 1 h, 2 h, 4 h, 8 h, 12 h, 24 h, 36 h, 48 h, 60 h, and 72 h for those treated with Banamine®-S Injectable Solution. Plasma samples were analyzed for flunixin concentration using an GLP-validated LC/MS-MS method. Swine plasma (K₂EDTA) is extracted by protein precipitation with an acetonitrile solution of the internal standard (Flunixin-d₃). The samples are then centrifuged, and the resulting supernatants are analyzed by LC-MS/MS using selective reaction monitoring in negative electrospray ionization mode. The ionic transitions monitored are m/z 295 to m/z 251 for Flunixin and m/z 298 to m/z 254 for the internal standard.

The following key pharmacokinetic parameters were calculated for each group (mean±standard error):

| Name | Unit | Buccal | Intravenous | Intramuscular | Perineum |
|---|---|---|---|---|---|
| AUC_{Last} | hour*ng/ | 34600.79 ± | 37808.06 ± | 26657.28 ± | 2078.39 ± |
| | mL | 4028.257 | 1610.4813 | 1844.2121 | 249.82561 |
| AUC_{∞} | hour*ng/ | 34703.96^{a} | 37971.76^{a} | 26778.74^{a} | 2303.471^{a} |
| | mL | | | | |
| % AUC_{∞(pred)} | % | 0.297285^{a} | 0.431122^{a} | 0.453551^{a} | 9.771378^{a} |
| t_{1/2} | hour | 11.55279^{a} | 6.616405^{a} | 6.665592^{a} | 23.35541^{a} |
| C_{Max} | ng/mL | 4172.5 ± | 7575 ± | 5230 ± | 147.575 ± |
| | | 472.48236 | 521.73588 | 313.55489 | 31.075939 |
| T_{Max} | hour | 2^{a} | 0.25^{a} | 0.25^{a} | 4^{a} |
| Cₗₐₛₜ | ng/mL | 6.19^{a} | 17.15^{a} | 12.63^{a} | 6.68^{a} |
| Tₗₐₛₜ | hour | 72^{a} | 48^{a} | 48^{a} | 60^{a} |
| r² | N/A | 0.998916^{a} | 0.999502^{a} | 0.998922^{a} | 0.939313^{a} |
| % F | % | 40^{a} | 100^{a} | 71^{a} | 3^{a} |

| Name | Ear Canal | Rectum | Midline |
|---|---|---|---|
| AUC_{Last} | 9393.675 ± | 14809.18 ± | 4326.05 ± |
| | 891.32057 | 5175.1178 | 656.8823 |
| AUC_{∞} | 9682.92^{a} | 14951.3^{a} | 4650.426^{a} |
| % AUC_{∞(pred)} | 2.987165^{a} | 0.950523^{a} | 6.975191^{a} |
| t_{1/2} | 15.27537^{a} | N/A^{b} | 18.24263^{a} |
| C_{Max} | 504.75 ± | 2079 ± | 157.15 ± |
| | 85.534959 | 861.6431 | 43.05991 |
| T_{Max} | 2^{a} | 2^{a} | 12^{a} |
| Cₗₐₛₜ | 13.125^{a} | 9.51^{a} | 12.325^{a} |
| Tₗₐₛₜ | 72^{a} | 72^{a} | 72^{a} |
| r² | 0.999727^{a} | 0.847996^{a} | 0.962754^{a} |
| % F | 11^{a} | 17^{a} | 5^{a} |

^{a}No standard error can be calculated for this parameter based on the nature of the sparse, non-compartmental analysis.
^{b}As the group r2 was ≤0.85, the group t½ is not reported.

The results generated showed that the highest exposure and bioavailability (% F) of flunixin was observed in the intramuscular group (71%). However, it was unexpected that the bioavailability of buccal administration (40%), was superior to the other modes of administration for flunixin transdermal solution. The bioavailabilities of these groups were rectum (17%), ear canal (11%), midline (5%), and perineum (3%).

The bioavailability observed with the midline application in swine (5%) was very disappointing when considering the bioavailability measured in bovine species with the same formulation (44%). However, the bioavailability observed with the buccal application (40%) was very promising. Moreover, the buccal application of flunixin has the advantage of being more convenient to administer for swine species than the other modes of administration (e.g. injectable, rectal, ear canal, perineum or midline).

Overall, these data support the development of the buccal administration of flunixin as the first pain medication to control the pain in swine undergoing several painful procedures, such as castration.

1. A method of treating pain in an animal comprising buccal administration to an animal in need thereof of a composition comprising flunixin or a salt thereof and a pharmaceutically acceptable carrier.

2. The method of claim 1, wherein the flunixin is flunixin meglumine.

3. The method of claim 1, wherein the pharmaceutically acceptable carrier comprises one or more penetration enhancers, an aprotic primary solvent or mixtures thereof.

4. The method of claim 3, wherein the penetration enhancers are menthol, propylene glycol dicaprylate/dicapratecaprate or mixtures thereof.

5. The method of claim 3, wherein the aprotic primary solvent is a pyrrolidone solvent.

6. The method of claim 3, wherein the aprotic primary solvent is 2-pyrollidone.

7. The method of claim 1, wherein the animal is a livestock animal.

8. The method of claim 7, wherein the livestock animal is a pig, a goat, a sheep or a bovid.

9. The method of claim 8, wherein the animal is a pig.

10. The method of claim 1, wherein the pain is a result of castration, tail docking or removal of needle teeth.

11. The method of claim 1, wherein the buccal administration is to the inside of the cheek of the animal.

12. A method of treating pain comprising buccal administration to an animal in need thereof a composition comprising.
a) flunixin meglumine,
b) 2-pyrrolindone,
c) menthol,
d) propylene glycol dicaprylate/dicapratecaprate, and
e) glyceryl monocaprylate.

13. The method of claim 12, wherein the animal is a pig.
